(19) United States
(12) Patent Application Publication
(10) Pub. No.: US 2026/0198882 A1
(43) Pub. Date: Jul. 16, 2026

(54) X-RAY CT APPARATUS AND CONTROL METHOD

(71) Applicants: KEIO UNIVERSITY, Tokyo (JP); Canon Kabushiki Kaisha, Tokyo (JP)

(72) Inventors: Yoshitake YAMADA, Tokyo (JP); Masahiro JINZAKI, Tokyo (JP); Minoru YAMADA, Hino (JP); Yoichi YOKOYAMA, Tokyo (JP); Ryotaro TANAKA, Nasushiobara (JP)

(73) Assignees: KEIO UNIVERSITY, Tokyo (JP); Canon Kabushiki Kaisha, Tokyo (JP)

(21) Appl. No.: 19/443,195

(22) Filed: Jan. 8, 2026

(30) Foreign Application Priority Data
Jan. 15, 2025 (JP) 2025-005509

(51) Int. Cl. A61B 6/00; A61B 6/03; A61B 6/04

(52) U.S. Cl. CPC A61B 6/54; A61B 6/032; A61B 6/0407; A61B 6/0487; A61B 6/4447

(57) ABSTRACT

An X-ray CT apparatus includes a gantry, a couch, and a control unit. The gantry including a scanner and a stand. The couch including a top plate and a support frame configured to support the top plate. The control unit configured to perform positioning control such that for executing first imaging which uses the couch, the scanner and the support frame are placed in a first positional relationship, and for executing second imaging which does not use the couch, the scanner and the support frame are placed in a second positional relationship. In the first positional relationship, a part of the support frame is located in a movable range of the scanner. In the second positional relationship, the support frame is located outside the movable range of the scanner.

### CROSS-REFERENCE TO RELATED APPLICATION

This application is based upon and claims the benefit of priority from prior Japanese Patent Application No. 2025-005509, filed January 15, 2025, the entire contents of which are incorporated herein by reference.

### FIELD

Embodiments described herein relate generally to an X-ray CT apparatus and a control method.

### BACKGROUND

An X-ray computed tomography (CT) apparatus for common use in a standing position and in a sitting position can switch, by inclining (tilting) a scanner by 90°, an imaging mode between a standing position imaging mode in which a subject in a standing position can be imaged or a sitting position imaging mode in which the subject in a sitting position can be imaged, and a recumbent position imaging mode in which the subject in a recumbent position lying on a couch can be imaged.

In this CT apparatus, at a time of switching from the recumbent position imaging mode to the standing position imaging mode or the sitting position imaging mode, there is a risk of interference between the couch and the scanner. It is thus necessary to install the couch at a distance from the scanner, thereby to prevent interference between the scanner and the couch. On the other hand, as the distance between the couch and the scanner becomes greater, the movable range (stroke) of a top plate needs to be made longer, and there is a risk of deterioration in imaging position accuracy due to the influence of bending of the top plate caused by the load of the subject.

### BRIEF DESCRIPTION OF THE DRAWINGS

FIG. 1 is a diagram illustrating a configuration example of an X-ray CT apparatus according to an embodiment.

FIG. 2 is a perspective view illustrating a state of the X-ray CT apparatus in a recumbent position imaging mode.

FIG. 3 is a diagram illustrating the X-ray CT apparatus in the recumbent position imaging mode, as viewed from above.

FIG. 4 is a perspective view illustrating a state of the X-ray CT apparatus in a standing position imaging mode.

FIG. 5 is a perspective view illustrating a state of the X-ray CT apparatus in a sitting position imaging mode.

FIG. 6 is a diagram illustrating the X-ray CT apparatus in the standing position imaging mode and the sitting position imaging mode, as viewed from above.

FIG. 7 is a flowchart exemplarily illustrating a procedure of a first mode switching process by the X-ray CT apparatus according to the embodiment.

FIG. 8 is a diagram illustrating the X-ray CT apparatus at a time of starting the first mode switching

process, as viewed from above.

FIG. 9 is a diagram illustrating a state in which the couch is moved from the state illustrated in FIG. 8.

FIG. 10 is a diagram illustrating a state in which the scanner is tilted from the state illustrated in FIG. 9.

FIG. 11 is a flowchart exemplarily illustrating a procedure of a second mode switching process by the X-ray CT apparatus according to the embodiment.

FIG. 12 is a diagram illustrating the X-ray CT apparatus at a time of starting the second mode switching process, as viewed from above.

FIG. 13 is a diagram illustrating a state in which the scanner is tilted from the state illustrated in FIG. 12.

FIG. 14 is a diagram illustrating a state in which the couch is moved from the state illustrated in FIG. 13.

FIG. 15 is a diagram illustrating an X-ray CT apparatus in a recumbent position imaging mode according to a first modification, as viewed from above.

FIG. 16 is a diagram illustrating the X-ray CT apparatus in a standing position imaging mode and a sitting position imaging mode according to the first modification, as viewed from above.

FIG. 17 is a diagram illustrating an X-ray CT apparatus in a recumbent position imaging mode according to a third modification, as viewed from above.

FIG. 18 is a diagram illustrating the X-ray CT apparatus in a standing position imaging mode and a sitting position imaging mode according to the third modification, as viewed from above.

FIG. 19 is a perspective view illustrating a state of an X-ray CT apparatus in a recumbent position imaging mode according to a fourth modification.

FIG. 20 is a perspective view illustrating a state of the X-ray CT apparatus in a standing position imaging mode and a sitting position imaging mode according to the fourth modification.

FIG. 21 is a diagram illustrating an X-ray CT apparatus in a recumbent position imaging mode according to a fifth modification, as viewed from above.

FIG. 22 is a diagram illustrating the X-ray CT apparatus in a standing position imaging mode and a sitting position imaging mode according to the fifth modification, as viewed from above.

FIG. 23 is a diagram illustrating an X-ray CT apparatus in a recumbent position imaging mode according to a sixth modification, as viewed from above.

FIG. 24 is a diagram illustrating the X-ray CT apparatus in a standing position imaging mode and a sitting position imaging mode according to the sixth modification, as viewed from above.

### DETAILED DESCRIPTION

In general, an X-ray CT apparatus includes a gantry, a couch, and a control unit. The gantry including a scanner with an imaging system, and a stand configured to tiltably

support the scanner. The couch including a top plate on which a subject is placed and a support frame configured to support the top plate so as to enable the top plate to move along a longitudinal direction. The control unit configured to perform positioning control such that for executing first imaging which uses the couch, the scanner and the support frame are placed in a first positional relationship, and for executing second imaging which does not use the couch, the scanner and the support frame are placed in a second positional relationship. In the first positional relationship, a part of the support frame is located in a movable range of the scanner. In the second positional relationship, the support frame is located outside the movable range of the scanner.

Hereinafter, embodiments of an X-ray CT apparatus and a control method will be described in detail with reference to the accompanying drawings. In the embodiments described below, elements denoted by identical reference signs are assumed to perform the same operations, and redundant descriptions thereof will be omitted as appropriate. Hereinafter, an embodiment will be described with reference to the accompanying drawings.

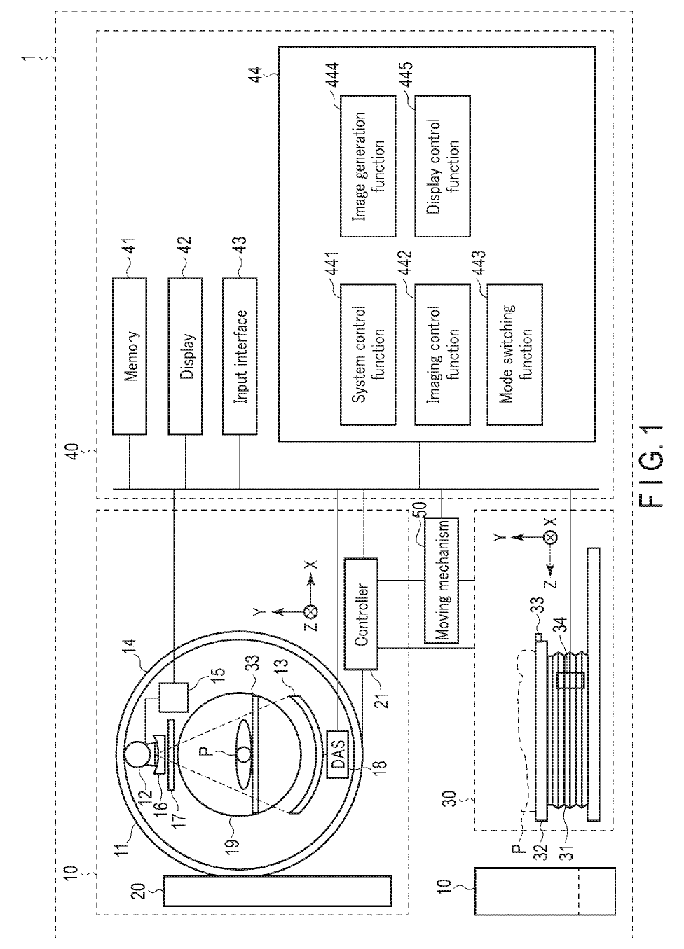

FIG. 1 is a diagram illustrating an example of a configuration of an X-ray CT apparatus 1 according to an embodiment. As illustrated in FIG. 1, the X-ray CT apparatus 1 includes a gantry 10, a couch 30, and a console 40. FIG. 1 illustrates a plurality of gantries 10 for the convenience of explanation, but the actual number of gantries may be one or two or more. The gantry 10 is a scan device having a configuration for performing X-ray CT imaging on a subject P such as a patient. The couch 30 is a carrier device on which the subject P to be subjected to X-ray CT imaging is placed and which regulates the position of the subject P. The console 40 is a computer that controls the gantry 10. For example, the gantry 10 and the couch 30 are installed in a CT examination room, and the console 40 is installed in a control room adjacent to the CT examination room. The gantry 10, the couch 30, and the console 40 are communicably connected to one another wirelessly or by wire. The console 40 need not necessarily be installed in the control room. For example, the console 40 may be installed together with the gantry 10 and the couch 30 in the same room. Alternatively, the console 40 may be incorporated into the gantry 10.

As illustrated in FIG. 1, the gantry 10 includes a scanner 11, an X-ray tube 12, an X-ray detector 13, a rotational frame 14, an X-ray high-voltage device 15, a wedge 16, a collimator 17, and data acquisition circuitry (data acquisition system: DAS) 18, an opening 19, a support column 20, and a controller 21. The scanner 11 is a substantially cylindrical structure in which the opening 19 is formed. The scanner 11 accommodates the X-ray tube 12 and the X-ray detector 13 that are disposed to be opposed to each other, with the opening 19 being interposed.

The scanner 11 and the support column 20 are connected to each other via a tilting mechanism and an ascending and descending mechanism. The support column 20 is an example of a stand that tiltably supports the scanner 11 via the tilting mechanism. The tilting mechanism is a mechanism for rotating the scanner 11. For example, as the tilting mechanism, a general rotating mechanism formed of a gear, a conveyor, and the like may be employed. The ascending and descending mechanism is a mechanism for moving the scanner 11 up and down along the vertical direction. For example, as the ascending and descending mechanism, a general linear motion mechanism such as a rack and pinion mechanism may be employed. Although an example in which one support column 20 is provided is illustrated here, two support columns 20 may be provided. In this case, the two support columns 20 are disposed to be opposed to each other, with the scanner 11 being interposed, and the scanner 11 is tiltably supported by the two support columns 20.

Here, a direction extending from a center axis of the opening 19 of the scanner 11 toward the support column 20 is defined as an X-axis direction, and directions perpendicular to the X-axis direction are defined as a Y-axis direction and a Z-axis direction. The X-axis direction is a direction substantially parallel to a rotational axis (hereinafter referred to as "tilt axis") of the scanner 11 by the tilting mechanism. The Y-axis direction is substantially parallel to the vertical direction and is also referred to as an up-and-down direction. The Z-axis direction is a direction perpendicular to the X-axis direction and Y-axis direction. In the present embodiment, the Z-axis direction is an example of a moving direction.

The X-ray tube 12 emits X-rays to the subject P. Specifically, the X-ray tube 12 includes a cathode that generates thermoelectrons, an anode that generates X-rays by receiving the thermoelectrons travelling from the cathode, and a vacuum tube that holds the cathode and the anode. The X-ray tube 12 is connected to the X-ray high-voltage device 15 via a high voltage cable. The X-ray high-voltage device 15 applies a tube voltage between the cathode and the anode. Thermoelectrons travel from the cathode to the anode upon application of the tube voltage. Tube current flows as thermoelectrons travel from the cathode to the anode. X-rays are generated through collision of the thermoelectrons with the anode.

The X-ray detector 13 detects the X-rays that have been emitted from the X-ray tube 12 and have passed through the subject P, and outputs an electric signal corresponding to the detected X-ray dose to the DAS 18. The X-ray detector 13 has a structure in which a plurality of X-ray detection element rows are aligned in a slice direction (row direction), each of the X-ray detection element rows including a plurality of X-ray detection elements aligned in a channel direction. The X-ray detector 13 is, for example, an indirect conversion-type detector including a grid, a scintillator array, and an optical sensor array. The scintillator array includes a plurality of scintillators. The scintillator outputs an amount of light corresponding to an amount of incident X-rays. The grid is arranged on the X-ray incident surface side of the scintillator array, and includes an X-ray shielding plate that absorbs scattered X-rays. The grid may be referred to as a "collimator (one-dimensional collimator or two-dimensional collimator)". The optical sensor array converts the light to an electric signal corresponding to the amount of light output from the scintillator. For example, a photodiode is used as the optical sensor.

Note that the X-ray detector 13 may be a photon-counting detector.

In the case of a photon-counting detector, the scintillator converts the incident X-rays into photons, the number of which corresponds to the intensity of the incident X-rays. The optical sensor array has the function of amplifying the light received from the scintillator and converting the amplified light into an electric signal, and generating an output signal (energy signal) having a peak value corresponding to the energy of the incident X-rays.

In addition, the X-ray detector 13 may be a direct conversion-type detector with a semiconductor element that converts incident X-rays into an electric signal.

The rotational frame 14 is an annular frame that supports the X-ray tube 12 and the X-ray detector 13 rotatably about the center axis of the opening 19. Specifically, the rotational frame 14 supports the X-ray tube 12 and the X-ray detector 13 in such a manner that the X-ray tube 12 and the X-ray detector 13 face each other. In addition to the X-ray tube 12 and the X-ray detector 13, the rotational frame 13 further supports the X-ray high-voltage device 15 and the DAS 18. The rotational frame 14 is supported by a stationary frame (not illustrated) so as to be rotatable around the center axis of the opening 19. A rotation mechanism includes, for example, a motor that generates a rotational drive force and a bearing that transmits the rotational drive force to the rotational frame 14 to rotate the rotational frame 14. The motor is provided to the stationary frame, and the bearing is physically connected to the rotational frame 13 and the motor, so that the rotational frame 14 rotates in accordance with the rotational force of the motor. By the rotation of the rotational frame 14, the X-ray tube 12 and the X-ray detector 13 rotate around the center axis of the opening 19. The rotational frame 14 is an example of a rotational unit.

The X-ray high-voltage device 15 includes a high-voltage generator and an X-ray controller. The high-voltage generator includes electric circuitry, such as a transformer and a rectifier, and generates high voltage to be applied to the X-ray tube 12, and filament current to be supplied to the X-ray tube 12. The X-ray controller controls output voltage in accordance with the X-rays emitted by the X-ray tube 12. The high-voltage generator may adopt a transformer system or an inverter system. The X-ray high-voltage device 15 may be provided to the rotational frame 14 or provided to the stationary frame (not illustrated) in the scanner 11.

The wedge 16 adjusts the dose of X-rays emitted to the subject P. Specifically, the wedge 16 attenuates the X-rays so that the dose of X-rays emitted from the X-ray tube 12 to the subject P exhibits a predetermined distribution. For example, a metal plate made of aluminum or the like, such as a wedge filter or a bow-tie filter, is used as the wedge 16.

The collimator 17 limits the range of applying X-rays that have passed through the wedge 16. The collimator 17 slidably supports a plurality of lead plates that shield X-rays and adjusts the shape of slits formed by the lead plates. The collimator 17 may be referred to as an X-ray diaphragm.

The DAS 18 reads from the X-ray detector 13 electric signals corresponding to the dose of X-rays detected by the X-ray detector 13. The DAS 18 amplifies the read electric signals and integrates the electric signals during a view period, thereby acquiring detection data with a digital value corresponding to the dose of X-rays during the view period. The detection data is also referred to as projection data. The DAS 18 is implemented by, for example, an application specific integrated circuit (ASIC) equipped with a circuit element capable of generating projection data. The projection data is transmitted to the console 40 via a non-contact data transmitter or the like.

Non-contact or contact-type communication circuitry is provided to each of the rotational frame 14 and the stationary frame, and the communication circuitry enables communication between the units supported by the rotational frame 14 and the stationary frame or an external apparatus of the scanner 11. For example, if optical communication is adopted as a non-contact communication method, detection data generated by the DAS 18 is transmitted, via optical communication, from a transmitter with a light-emitting diode (LED), which is provided to the rotational frame 14, to a receiver with a photodiode, which is provided to the stationary frame of the scanner 11, and further transferred from the stationary frame to the console 40 by the transmitter. As the communication method, not only the aforementioned communication methods but also a non-contact data transmission method such as a capacitive coupling method and a radio wave method, and a contact-type data transmission method using a slip ring and an electrode brush may be adopted.

The controller 21 controls the X-ray high-voltage device 15 or the DAS 18 to perform X-ray CT imaging in accordance with an imaging control function 442 of processing circuitry 44 of the console 40. The controller 21 includes processing circuitry including a central processing unit (CPU), a micro processing unit (MPU), or the like, and a drive mechanism such as a motor or an actuator. The processing circuitry includes, as hardware resources, a processor such as a CPU or the like and a memory such as a read only memory (ROM), a random access memory (RAM), or the like. The controller 21 implements various functions via a processor executing programs loaded into a memory. Note that the various functions may not be implemented by single processing circuitry. The processing circuitry may be constituted by combining a plurality of independent processors, which execute respective programs to implement the respective functions. The controller 21 may be realized by an ASIC or a field programmable gate array (FPGA). The controller 21 may also be realized by a complex programmable logic device (CPLD) or a simple programmable logic device (SPLD).

In addition, the controller 21 functions to control the operation of the scanner 11 and the couch 30 upon receiving an input signal from an input interface 43 (to be described later) or a control signal from the processing circuitry 44. The input interface 43 is attached to, for example, the console 40 or the scanner 11. For example, the controller 21 performs control to rotate the rotational frame 14, control to tilt the scanner 11, and control to move the couch 30 and the top plate 33. The controller 21 may be provided to the scanner 11 or the console 40.

The couch 30 includes a base 31, a support frame 32, the top plate 33, and a couch drive 34. The base 31 is installed on the floor. The base 31 is a housing that supports the support frame 32 movably in a direction perpendicular to the floor. The support frame 32 is a frame provided on top of the base 31. The support frame 32 supports the top plate 33 slidably along an imaging direction (along a longitudinal direction) of recumbent position imaging. The subject P in a recumbent position is placed on the top plate 33.

The couch drive 34 is housed in the housing of the couch 30. The couch drive 34 is a motor or actuator that generates driving force to move the top plate 33, on which the subject P is placed, and the support frame 32. The couch drive 34 operates in accordance with the control performed by the processing circuitry 44, the console 40, and the like.

Hereinafter, a description is given of imaging modes that the X-ray CT apparatus 1 of the present embodiment executes.

The X-ray CT apparatus 1 includes a recumbent position imaging mode in which the subject P in a recumbent position is imaged, a standing position imaging mode in which the subject P in a standing position is imaged, and a sitting position imaging mode in which the subject in a sitting position is imaged. The recumbent position imaging is an example of first imaging that uses the couch 30, and the positional relationship between the gantry 10 and the support frame 32 of the couch 30 in the recumbent position imaging mode is an example of a first positional relationship in which the first imaging is executed. The standing position imaging and the sitting position imaging are examples of second imaging that does not use the couch 30, and the positional relationship between the gantry 10 and the support frame 32 of the couch 30 in the standing position imaging mode and the sitting position imaging mode is an example of a second positional relationship in which the second imaging is executed.

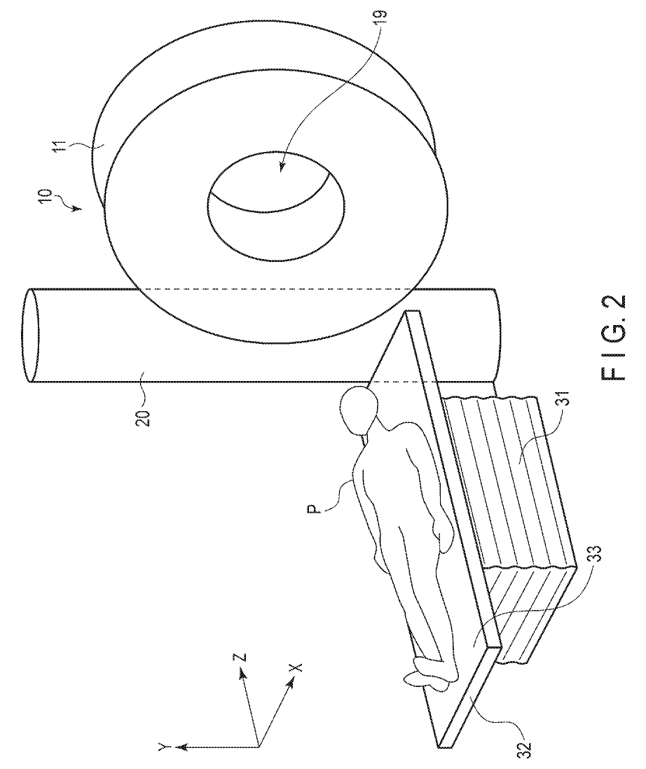

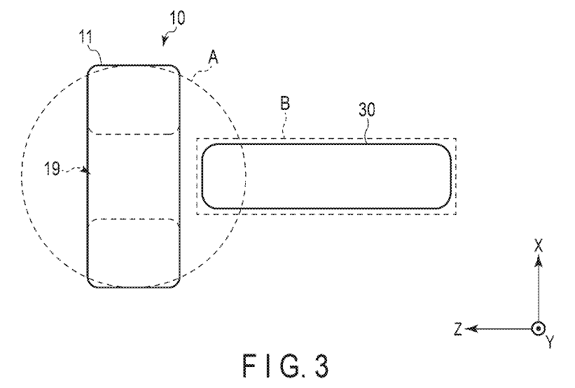

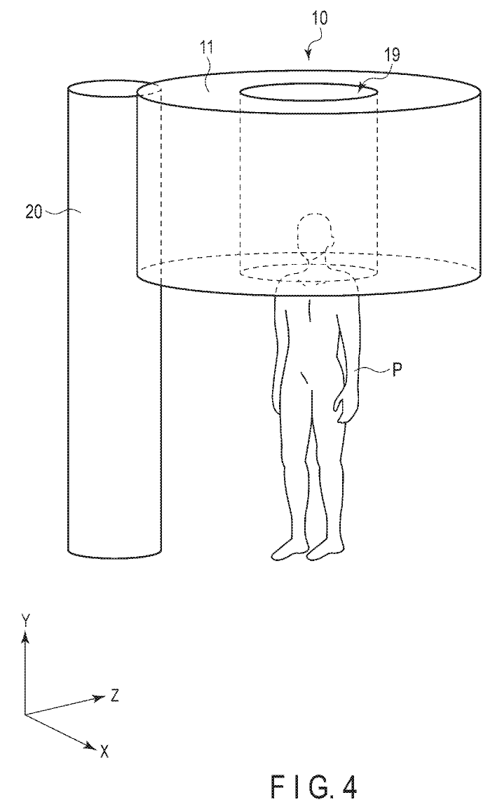

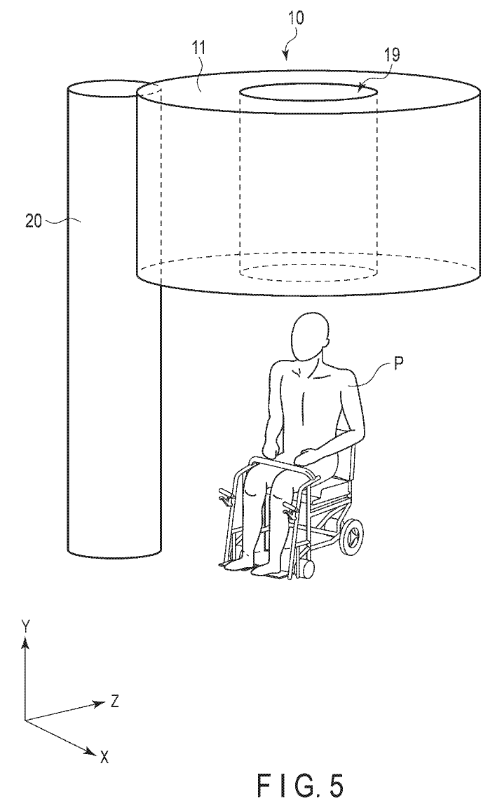

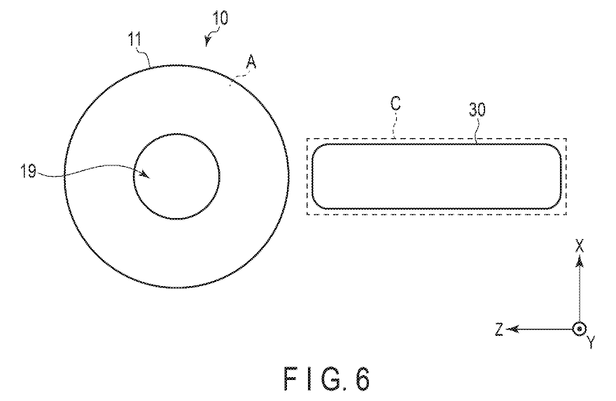

The states of the scanner 11 and the couch 30 in the respective imaging modes are described. FIG. 2 is a perspective view illustrating the positional relationship between the scanner 11 and the couch 30 in the recumbent position imaging mode. This positional relationship is an example of the first positional relationship. FIG. 3 is a diagram illustrating the scanner 11 and the couch 30 in the recumbent position imaging mode, as viewed from above. FIG. 4 is a perspective view illustrating the positional relationship of the scanner 11 in the standing position imaging mode. FIG. 5 is a perspective view illustrating the positional relationship of the scanner 11 in the sitting position imaging mode. The positional relationships of FIG. 4 and FIG. 5 are examples of the second positional relationship. FIG. 6 is a diagram illustrating the scanner 11 and the couch 30 in the standing position imaging mode and the sitting position imaging mode, as viewed from above. In FIG. 4 and FIG. 5, an illustration of the couch 30 is omitted.

As illustrated in FIG. 2 and FIG. 3, in the recumbent position imaging mode, the scanner 11 is fixed in a state in which the center axis of the opening 19 is substantially parallel to the Z-axis direction. At this time, the couch 30 is moved to an imaging position B. The subject P lies on the top plate 33 in the recumbent position. In this state, the top plate 33 is inserted into the opening 19 along the Z-axis direction, and thereby imaging is executed. The imaging position B is preset at a position that is distanced from the scanner 11 in the Z direction in the vicinity of the scanner 11. In addition, the imaging position B is set at a position at which the top plate 33 can be inserted into the opening 19 and which includes a movable range A of the scanner 11. The movable range A of the scanner 11 is a range through which the scanner 11 passes in a horizontal direction (X-axis direction and Z-axis direction) at a time when the scanner 11 is tilted.

As illustrated in FIG. 4 and FIG. 6, in the standing position imaging mode, the scanner 11 is fixed in a state in which the center axis of the opening 19 is substantially parallel to the Y-axis direction (vertical direction). At this time, the couch 30 is moved to a retraction position C. The retraction position C is set at a position which is distant from the movable range A of the scanner 11 and is moved in a direction away from the scanner 11 in the Z-axis direction, relative to the imaging position B. In the state in which the standing subject P is positioned in the opening 19, imaging is executed by the scanner 11 being moved in the up-and-down direction by the ascending and descending mechanism.

As illustrated in FIG. 5 and FIG. 6, in the sitting position imaging mode, imaging is executed on the subject P that sits on a chair or a wheelchair. In the sitting position imaging mode, like the standing position imaging mode, the scanner 11 is fixed in a state in which the center axis of the opening 19 is substantially parallel to the Y-axis direction (vertical direction). At this time, the couch 30 is moved to the retraction position C. Imaging is executed by the scanner 11 being moved in the up-and-down direction by the ascending and descending mechanism in the state in which the subject P sitting on the chair or wheelchair is positioned in the opening 19.

As described above, the body axis direction of the subject is substantially orthogonal between the standing position imaging mode or sitting position imaging mode, and the recumbent position imaging mode. The Z-axis direction may be called an imaging direction of recumbent position imaging or a major-axis direction of the couch, and the X-axis direction may be called a width direction of the couch. The Y-axis direction may be called an imaging direction of standing position image and sitting position imaging.

Referring back to FIG. 1, the X-ray CT apparatus 1 further includes a moving mechanism 50. The moving mechanism 50 is connected to the couch 30 and moves the couch 30 along a predetermined moving direction. The moving mechanism 50 of the present embodiment moves the couch 30 along the major-axis direction (Z-axis direction, the imaging direction of recumbent position imaging). The moving mechanism 50 is, for example, a linear motion mechanism such as a motor or an actuator that generates driving force for moving the couch 30. The moving mechanism 50 may be constituted by a carriage that can run on a rail provided on the floor of the examination room along the Z-axis direction, and a linear motion mechanism that drives the carriage. The moving mechanism 50 is connected to the console 40 via the controller 21. The controller 21 includes a function of executing operational control of the moving mechanism 50 in accordance with the control of the console 40, and moves the couch 30 in a predetermined moving direction in accordance with the control of the console 40. By controlling the driving of the moving mechanism 50, the controller 21 executes control to set the couch 30 or the gantry 10 in an appropriate state in accordance with the imaging mode.

The console 40 includes a memory 41, a display 42, an input interface 43, and processing circuitry 44. Data communication between the memory 41, the display 42, the input interface 43, and the processing circuitry 44 is performed via a bus. The console 40 is described as being separate from the scanner unit 10, but the console 40, or some of the components of the console 40, may be included in the gantry 10.

The memory 41 is a storage device, such as a hard disk drive (HDD), a solid-state drive (SSD), or an integrated circuit storage device, which stores various types of information. Aside from an HDD, an SSD, or the like, the memory 41 may be a portable storage medium such as a compact disc (CD), a digital versatile disc (DVD), a Blu-ray (registered trademark) disc (BD), or a flash memory. Alternatively, the memory 41 may be a drive that reads and writes various types of information from and in, for example, a semiconductor memory device such as a flash memory or a RAM. Besides, the storage area of the memory 41 may be in the X-ray CT apparatus 1 or in an external storage device connected via a network.

The display 42 displays various types of information. Various types of displays may be discretionarily and suitably adopted as the display 42. For example, a liquid crystal display (LCD), a cathode ray tube (CRT) display, an organic electroluminescence display (OELD), or a plasma display can be used as the display 42. The display 42 may be provided in any place in the control room. The display 42 may be provided to the gantry 10. The display 42 may be a desktop-type display, or may be constituted by a tablet terminal or the like capable of performing wireless communication with the main body of the console 40. One, or two or more projectors may be used as the display 42.

The input interface 43 receives various input operations from a user, converts the received input operations into electric signals, and outputs the electric signals to the processing circuitry 44. The user is, for example, a doctor or an engineer, and may also be called an operator. For example, a mouse, a keyboard, a trackball, a switch, a button, a joystick, a touch pad, a touch panel display, or the like can be suitably used as the input interface 43. In the embodiment, the input interface 43 does not necessarily include physical operation components such as a mouse, a keyboard, a trackball, a switch, a button, a joystick, a touch pad, and a touch panel display. Examples of the input interface 43 include processing circuitry for electric signals, which receives an electric signal corresponding to an input operation from an external input device separate from its own apparatus, and outputs this electric signal to the processing circuitry 44. The input interface 43 may be provided to the gantry 10. Alternatively, the input interface 43 may be constituted by, for example, a tablet terminal capable of performing wireless communication with the main body of the console 40.

The processing circuitry 44 controls the operation of the entirety of the X-ray CT apparatus 1 in accordance with the electric signal of the input operation output from the input interface 43. The processing circuitry 44 generates image data, based on the electric signal output from the X-ray detector 13. For example, the processing circuitry 44 includes a processor, such as a CPU, an MPU, or a GPU, and a memory, such as a ROM or a RAM, as hardware resources. Through a processor that executes a program loaded into the memory, the processing circuitry 44 implements a system control function 441, an imaging control function 442, a mode switching function 443, an image generation function 444, and a display control function 445.

The respective functions are not necessarily implemented by single processing circuitry. The processing circuitry may be constituted by combining a plurality of independent processors that execute programs to implement the respective functions.

With the system control function 441, the processing circuitry 44 controls each unit of the X-ray CT apparatus 1 in accordance with the loaded control program. In addition, with the system control function 441, the processing circuitry 44 controls the driving of the couch 30 via the controller 21.

With the imaging control function 442, the processing circuitry 44 controls the X-ray high-voltage device 15, the controller 21, and the DAS 18 in accordance with imaging conditions, and performs X-ray CT imaging.

With the mode switching function 443, the processing circuitry 44 controls the ascending and descending mechanism and the tilting mechanism, and adjusts the state of the scanner 11 such that the scanner 11 has a position and attitude corresponding to the imaging mode. With the mode switching function 443, the processing circuitry 44 controls the moving mechanism 50 and moves the couch 30 to a proper position corresponding to the imaging mode. In this case, at a time of switching to an imaging mode that uses the couch 30, the processing circuitry 44 moves the couch 30 to a position where the couch 30 overlaps the movable range of the scanner 11. At a time of switching to an imaging mode that does not use the couch 30, the processing circuitry 44 moves the couch 30 to a position where the couch 30 does not overlap the movable range of the scanner 11. In other words, the processing circuitry 44 performs positioning control such that for executing first imaging which uses the couch, the scanner and the support frame are placed in a first positional relationship, and for executing second imaging which does not use the couch, the scanner and the support frame are placed in a second positional relationship.

For example, based on the reception of a signal for a transition from the recumbent position imaging mode to the standing position imaging mode or the sitting position imaging mode, the processing circuitry 44 tilts the scanner 11 and controls the moving mechanism 50 such that the scanner 11 and the couch 30 move away from each other. At this time, the processing circuitry 44 controls the moving mechanism 50 via the controller 21, and moves the couch 30 from the imaging position B to the retraction position C along the Z-axis direction.

The signal for the transition from the recumbent position imaging mode to the standing position imaging mode or the sitting position imaging mode is an example of a first signal for a transition from the first positional relationship to the second positional relationship. In addition, the control to move the couch 30 in a direction away from the scanner 11 along the Z-axis direction is an example of first control. Specifically, based on the first signal for the transition from the first positional relationship at the time of executing the first imaging using the couch 30 to the second positional relationship at the time of executing the second imaging not using the couch 30, the processing circuitry 44 executes, in the mode switching function 443, the first control that controls the moving mechanism 50 in such a manner that the gantry 10 and the couch 30 move away from each other. The processing circuitry 44 that executes the mode switching function 443 is an example of a control unit.

In addition, based on the reception of a signal for a transition from the standing position imaging mode or the sitting position imaging mode to the recumbent position imaging mode, the processing circuitry 44 tilts the scanner 11 and controls the moving mechanism 50 such that the scanner 11 and the couch 30 approach each other. At this time, the processing circuitry 44 controls the moving mechanism 50 via the controller 21, and moves the couch 30 from the retraction position C to the imaging position B along the Z-axis direction. Note that, with the mode switching function 443, the processing circuitry 44 can also acquire both of the first signal and the second signal, or at least of the first signal and the second signal, by receiving an examination order from an external device. For example, with the mode switching function 443, the processing circuitry 44 can acquire at least one of the first signal and the second signal, by receiving the examination order including the first signal or second signal indicating the transfer of the imaging mode, from a server or the like of a radiology information system (RIS).

The signal for the transition from the standing position imaging mode or the sitting position imaging mode to the recumbent position imaging mode is an example of a second signal for a transition from the second positional relationship to the first positional relationship. In addition, the control to move the couch 30 in a direction toward the scanner 11 along the Z-axis direction is an example of second control. Specifically, based on the second signal for the transition from the second positional relationship at the time of executing the second imaging not using the couch 30 to the first positional relationship at the time of executing the first imaging using the couch 30, the processing circuitry 44 executes, in the mode switching function 443, the second control that controls the moving mechanism 50 in such a manner that the gantry 10 and the couch 30 approach each other. The processing circuitry 44 that executes the mode switching function 443 is an example of the control unit.

Besides, with the mode switching function 443, the processing circuitry 44 receives the examination order including attitude information of the subject P, for example, from the server or the like of the RIS, and determines the necessity/nonnecessity of the transition of the imaging mode. Furthermore, with the mode switching function 443, the processing circuitry 44 can generate the first signal or the second signal, in a case where the transition of the imaging mode is determined to be necessary.

With the image generation function 444, the processing circuitry 44 subjects projection data related to the subject P to a reconstruction process and generates a CT image. A filter correction backprojection method or a successive approximation reconstruction method is used as the reconstruction process. A reconstruction process that incorporates a denoising process using machine learning into these methods may also be used as the reconstruction process. The processing circuitry 44 converts a CT image to a cross-sectional image of a given cross section or a rendering image in a given direction of a visual point. The conversion is performed based on an input operation received from the user through the input interface 43. For example, the processing circuitry 44 subjects the reconstructed image data to three-dimensional image processing such as volume rendering, surface volume rendering, pixel value projection processing, multiplanar reconstruction (MPR) processing, curved MPR (CPR) processing, or the like, and generates a rendering image in a given direction of a visual point.

With the display control function 445, the processing circuitry 44 causes a generated CT image and rendering image to be displayed, for example, on the display 42.

Note that the console 40 is described above such that a plurality of functions are performed with a single console, but a plurality of functions may be performed with separate consoles. The processing circuitry 44 is not limited to a case of being included in the console 40, and may be included in an integrated server that collectively performs processes on projection data acquired by a plurality of medical diagnostic imaging apparatuses. Post-processing may be performed by either the console 40 or an external workstation. The process may also be performed simultaneously by both the console 40 or the external workstation.

The X-ray CT apparatus 1 has various types such as a third-generation CT apparatus and a fourth-generation CT

apparatus, any of which is applicable to the present embodiment. The third-generation CT apparatus is of a "rotate/rotate-type", in which an X-ray tube and a detector integrally rotate around a subject. The fourth-generation CT apparatus is of a "stationary/rotate-type", in which a number of X-ray detection elements arrayed in a ring shape are fixed and only an X-ray tube rotates around a subject.

Although not illustrated, the X-ray CT apparatus 1 may include a communication interface. The communication interface is an interface that connects the X-ray CT apparatus 1 with a workstation, a picture archiving and communication system (PACS), a hospital information system (HIS), a radiology information system (RIS), etc., via a local area network (LAN), etc. The communication interface transmits and receives various kinds of information to and from the connected workstation, PACS, HIS, and RIS.

Next, the operation of the X-ray CT apparatus 1 according to the present embodiment is described.

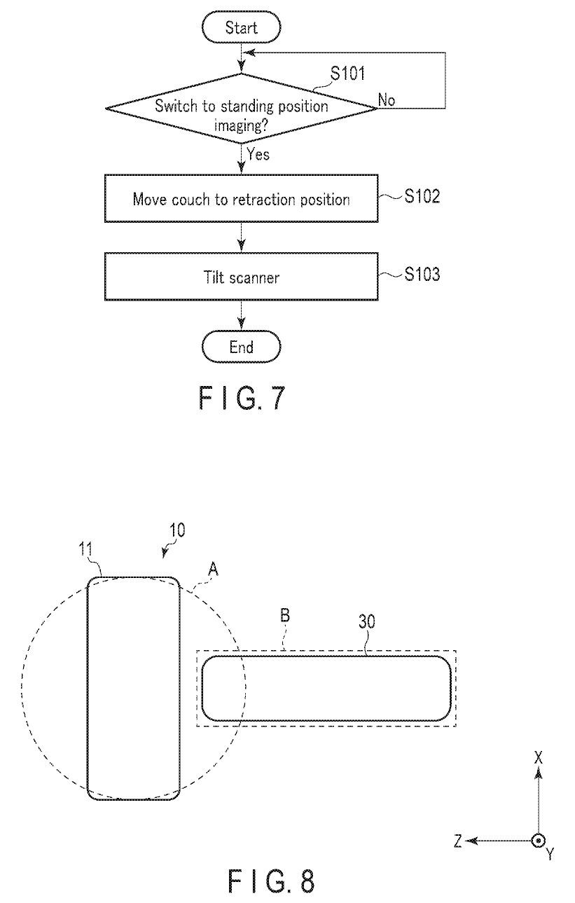

FIG. 7 is a flowchart illustrating an example of a procedure of a first mode switching process executed by the mode switching function 443 of the processing circuitry 44. The first mode switching process is a process that is executed at a time of switching the imaging mode of the X-ray CT apparatus 1 from the recumbent position imaging mode to the standing position imaging mode or the sitting position imaging mode. By way of example, a case is described in which the recumbent position imaging mode is changed to the standing position imaging mode, but the standing position imaging may be read as the sitting position imaging. The processing procedure to be described below is merely an example, and each process may be modified as much as possible. Moreover, in regard to the processing procedures to be described below, omission, replacement and addition of steps may be made as appropriate, in accordance with modes of implementation.

The first mode switching process is started in the state of the recumbent position imaging mode. FIG. 8 is a diagram illustrating the positional relationship between the scanner 11 and the couch 30 in the recumbent position imaging mode. In the recumbent position imaging mode, the center axis of the opening 19 of the scanner 11 is fixed in the state of extending in the horizontal direction (Z-axis direction), and the couch 30 is disposed in the imaging position B. This positional relationship is an example of the first positional relationship.

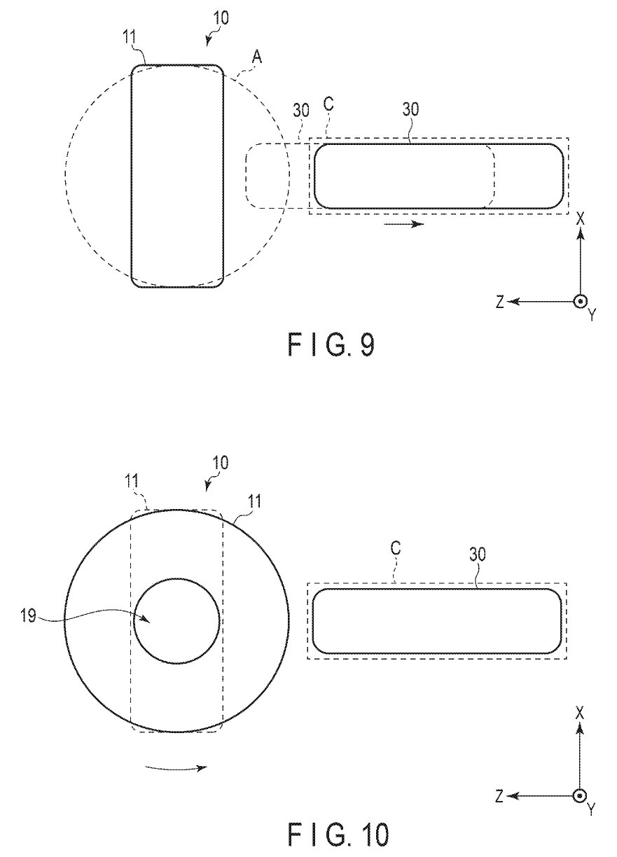

If an instruction to switch to the standing position imaging mode (step S101-Yes) is input, the processing circuitry 44 moves the couch 30 from the imaging position B to the retraction position C along the Z-axis direction (step S102). FIG. 9 is a diagram illustrating a state in which the couch 30 is moved from the state illustrated in FIG. 8 to the retraction position C. If the couch 30 moves to the retraction position C, the processing circuitry 44 tilts the scanner 11 by 90°, and fixes the scanner 11 in the state in which the center axis of the opening 19 extends in the vertical direction (Y-axis direction) (step S103). FIG. 10 is a diagram illustrating the positional relationship between the scanner 11 and the couch 30 at a time when the scanner 11 is tilted from the state illustrated in FIG. 9. This positional relationship is an example of the second positional relationship.

If the tilting of the scanner 11 ends, the subject moves into the inside of the opening 19, and the standing position imaging is executed by the up-and-down movement of the scanner 11. At this time, since the couch 30 has moved to the retraction position C that is distant from the movable range A of the scanner 11, the imaging can be performed without interference between the scanner 11 and the couch 30.

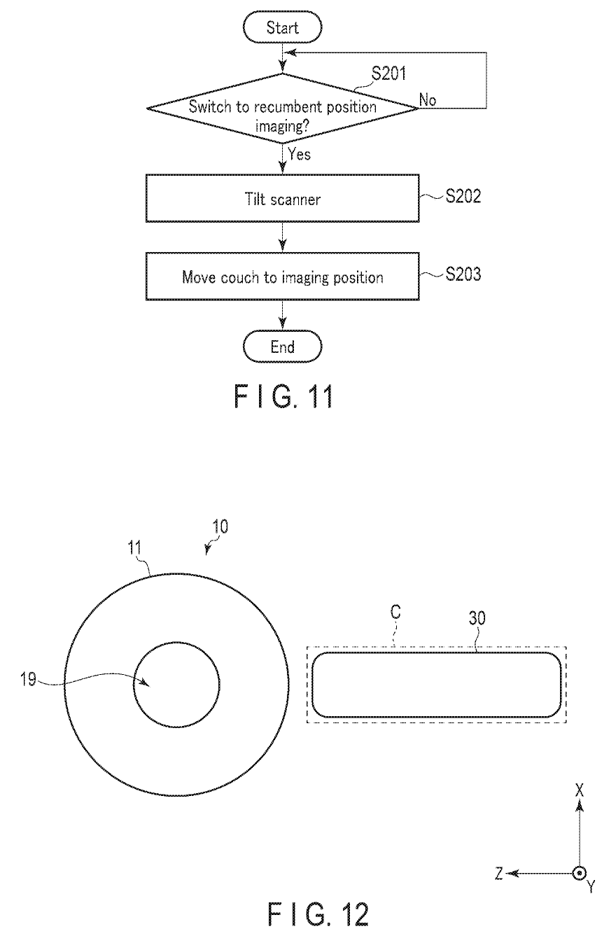

FIG. 11 is a flowchart illustrating an example of a procedure of a second mode switching process executed by the mode switching function 443 of the processing circuitry 44. The second mode switching process is a process that is executed at a time of switching the imaging mode of the X-ray CT apparatus 1 from the standing position imaging mode or the sitting position imaging mode to the recumbent position imaging mode. By way of example, a case is described in which the standing position imaging mode is switched to the recumbent position imaging mode, but the standing position imaging may be read as the sitting position imaging. The processing procedure to be described below is merely an example, and each process may be modified as much as possible. Moreover, in regard to the processing procedures to be described below, omission, replacement and addition of steps may be made as appropriate, in accordance with modes of implementation.

The second mode switching process is started in the state of the standing position imaging mode. FIG. 12 is a diagram illustrating the positional relationship between the scanner 11 and the couch 30 in the standing position imaging mode. In the standing position imaging mode, the center axis of the opening 19 of the scanner 11 is fixed in the state of extending in the vertical direction (Y-axis direction), and the couch 30 is disposed in the retraction position C. This positional relationship is an example of the second positional relationship.

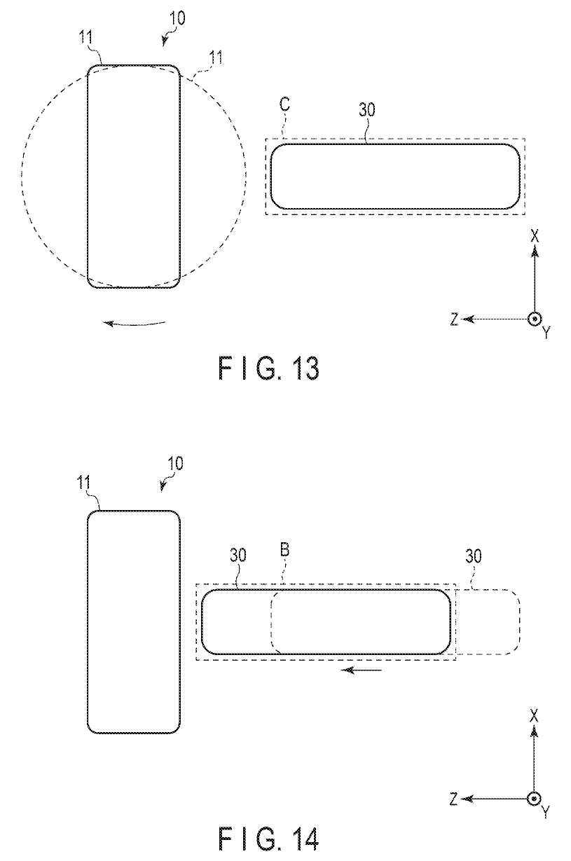

If an instruction to switch to the recumbent position imaging mode (step S201-Yes) is input, the processing circuitry 44 tilts the scanner 11 by 90° from the state in which the center axis of the opening 19 extends in the vertical direction (Y-axis direction), and fixes the scanner 11 in the state in which the center axis of the opening 19 extends in the horizontal direction (Z-axis direction) (step S202). FIG. 13 is a diagram illustrating a state in which the scanner 11 is tilted from the state illustrated in FIG. 12. If the tilting of the scanner 11 ends, the processing circuitry 44 moves the couch 30 from the retraction position C to the imaging position B along

the Z-axis direction (step S203). FIG. 14 is a diagram illustrating the positional relationship between the scanner 11 and the couch 30 at a time when the couch 30 has moved to the imaging position B from the state illustrated in FIG. 13. This positional relationship is an example of the first positional relationship.

If the moving of the couch 30 ends, the top plate 33 on which the subject is placed is conveyed into the opening 19 of the scanner 11, and the recumbent position imaging is performed. At this time, since the couch 30 is moved to the imaging position B adjacent to the movable range A of the scanner 11, the imaging can be performed while suppressing the sagging of the top plate 33.

The X-ray CT apparatus 1 of the present embodiment includes the gantry 10 including the scanner 11 and the support column 20; the couch 30 including the top plate 33; the moving mechanism 50; and the console 40. The moving mechanism 50 moves the couch 30 in the Z-axis direction. Based on the signal for tilting the scanner 11 from the recumbent position imaging mode to the standing position imaging mode or the sitting position imaging mode, the console 40 controls the moving mechanism 50 in such a manner that the gantry 10 and the couch 30 move away from each other. In this case, after moving the couch 30 in the direction away from the gantry 10, the console 40 tilts the scanner 11.

By the above-described configuration, according to the X-ray CT apparatus 1 of the present embodiment, the couch 30 can automatically be moved to the retraction position C that is distant from the scanner 11, at a time of performing the standing position imaging or the sitting position imaging with no use of the couch 30, from the state of the recumbent imaging mode using the couch 30. Thereby, without the user such as a doctor or an engineer moving the couch 30 by manual operation, the interference between the scanner 11 and the couch 30 can be prevented in the standing position imaging or sitting position imaging.

In addition, based on the signal for tilting the scanner 11 from the standing position imaging mode or the sitting position imaging mode to the recumbent position imaging mode, the console 40 controls the moving mechanism 50 in such a manner that the gantry 10 and the couch 30 approach each other. In this case, after tilting the scanner 11, the console 40 moves the couch 30 in the direction toward the gantry 10.

By the above-described configuration, according to the X-ray CT apparatus 1 of the present embodiment, the couch 30 can automatically be moved to the imaging position B near the scanner 11, at a time of performing the recumbent position imaging using the couch 30, from the state of the standing position imaging mode or the sitting position imaging mode with no use of the couch 30. Thereby, without the user such as a doctor or an engineer moving the couch 30 by manual operation, the deterioration in position accuracy due to the sagging of the top plate 33 in the recumbent position imaging can be prevented.

### First Modification

In the above-described embodiment, the example was described in which the couch 30 is moved along the major-axis direction (Z-axis direction) at the time of switching the imaging mode. In the present modification, in accordance with the imaging mode, the couch 30 is moved along the width direction (X-axis direction) of the couch 30. The moving mechanism 50 of the present modification moves the couch 30 along the width direction (X-axis direction) of the couch 30.

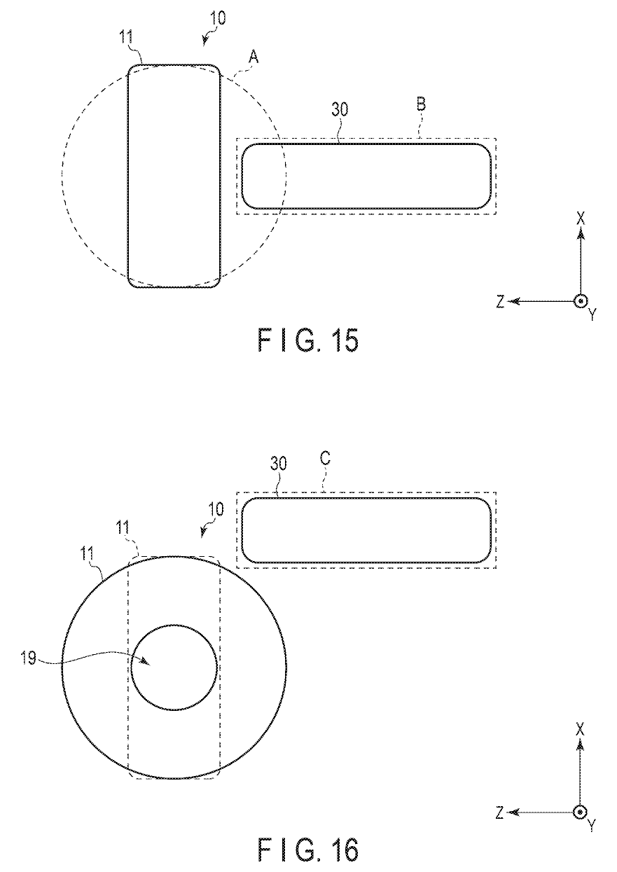

FIG. 15 is a diagram illustrating the positional relationship between the scanner 11 and the couch 30 in the recumbent position imaging mode, as viewed from above. FIG. 16 is a diagram illustrating the positional relationship between the scanner 11 and the couch 30 in the standing position imaging mode or the sitting position imaging mode, as viewed from above. In the standing position imaging mode or the sitting position imaging mode, the couch 30 is moved to the retraction position C. The retraction position C is set at a position that is distant from the movable range A of the scanner 11 and that is moved away from the scanner 11 in the X-axis direction, relative to the imaging position B. This positional relationship is an example of the second positional relationship.

In the present modification, in the recumbent position imaging mode, if an instruction to switch to the standing position imaging mode or the sitting position imaging mode is input, the scanner 11 is tilted after moving the couch 30 from the imaging position B to the retraction position C along the X-axis direction. In addition, in the standing position imaging mode or the sitting position imaging mode, if an instruction to switch to the recumbent position imaging mode is input, the scanner 11 is tilted and thereafter the couch 30 is moved from the retraction position C to the imaging position B along the X-axis direction.

### Second Modification

In accordance with the imaging mode, the couch 30 may be moved along the vertical direction (Y-axis direction). In the present modification, the vertical direction (Y-axis direction) is the moving direction. The moving mechanism 50 moves the couch 30 along the vertical direction (Y-axis direction). The retraction position is set at a position that is distant from the movable range A of the scanner 11 and that is moved away from the scanner 11 in the Y-axis direction, relative to the imaging position. The retraction position may be set on the upper side of the imaging position or may be set on the lower side of the imaging position. The retraction position may be set on the ceiling or in the inside of the floor. As the moving mechanism 50, use is made of a mechanism that is fixed on the ceiling and suspends the couch 50, or a carry-in mechanism that carries the couch 30 into the space provided in advance under the floor.

In the present modification, in the recumbent position imaging mode, if an instruction to switch to the standing position imaging mode or the sitting position imaging mode is input, the scanner 11 is tilted after moving the couch 30 from the imaging position to the retraction position along the Y-axis direction. In addition, in the standing position imaging mode or the sitting position imaging mode, if an instruction to switch to the recumbent position imaging mode is input, the scanner 11 is tilted and thereafter the couch 30 is moved from the retraction position to the imaging position along the Y-axis direction.

### Third Modification

In accordance with the imaging mode, the couch 30 may be rotated around a rotational axis extending in the vertical direction (Y-axis direction). In the present modification, the rotational direction around the rotational axis is the moving direction.

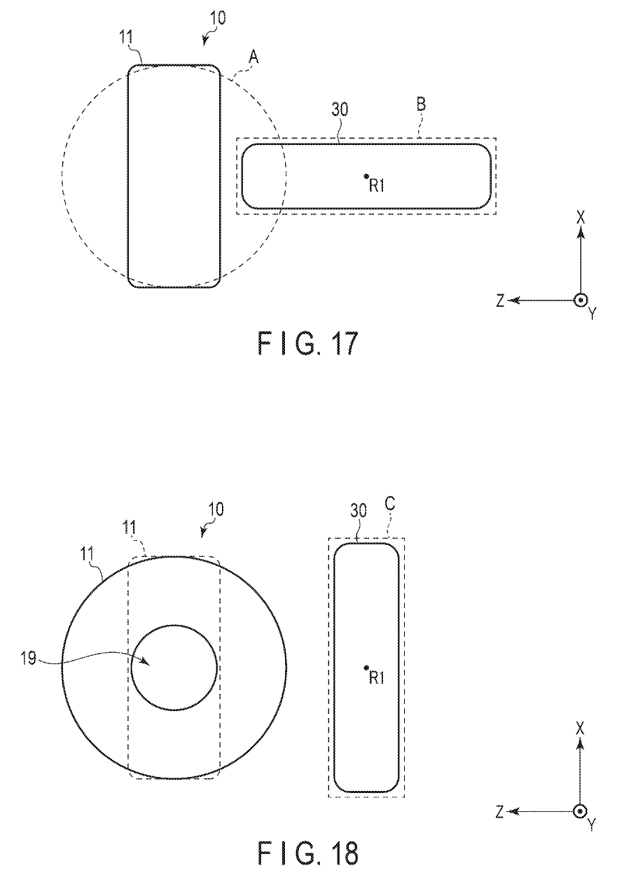

FIG. 17 is a diagram illustrating the positional relationship between the scanner 11 and the couch 30 in the recumbent position imaging mode, as viewed from above. FIG. 18 is a diagram illustrating the positional relationship between the scanner 11 and the couch 30 in the standing position imaging mode or the sitting position imaging mode, as viewed from above. In the standing position imaging mode or the sitting position imaging mode, the couch 30 is moved to the retraction position C. The retraction position C is set at a position which is distant from the movable range A of the scanner 11 and at which the couch 30 is rotated by 90°, relative to the imaging position B, around a rotational axis R1 that is substantially parallel to the vertical direction. This positional relationship is an example of the second positional relationship. The moving mechanism 50 is attached to the couch 30, and rotates the couch 30 around the rotational axis R1.

In the present modification, in the recumbent position imaging mode, if an instruction to switch to the standing position imaging mode or the sitting position imaging mode is input, the scanner 11 is tilted after rotating the couch 30 around the rotational axis R1 and moving the couch 30 from the imaging position B to the retraction position C. In addition, in the standing position imaging mode or the sitting position imaging mode, if an instruction to switch to the recumbent position imaging mode is input, the scanner 11 is tilted and thereafter the couch 30 is rotated around the rotational axis R1 and is moved from the retraction position C to the imaging position B along the X-axis direction.

### Fourth Modification

In accordance with the imaging mode, the couch 30 may be rotated around a rotational axis that is inclined with respect to the major-axis direction (Z-axis direction) of the couch 30 and the vertical direction (Y-axis direction). In the present modification, the rotational direction around the rotational axis is the moving direction.

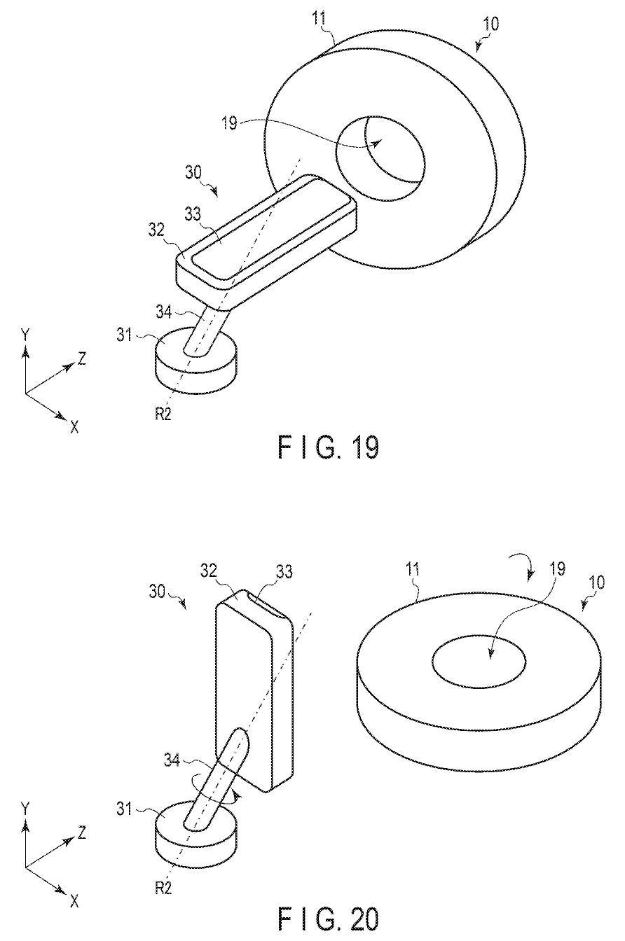

FIG. 19 is a perspective view illustrating the positional relationship between the scanner 11 and the couch 30 in the recumbent position imaging mode. FIG. 20 is a perspective view illustrating the positional relationship between the scanner 11 and the couch 30 in the standing position imaging mode or the sitting position imaging mode. In the standing position imaging mode or the sitting position imaging mode, the couch 30 is moved to the retraction position C. The retraction position C is set at a position which is distant from the movable range A of the scanner 11 and at which the couch 30 is rotated by 180°, relative to the imaging position B, around a rotational axis R2. This positional relationship is an example of the second positional relationship. The moving mechanism 50 is attached to the couch 30, and rotates the couch 30 around the rotational axis R2.

In the present modification, in the recumbent position imaging mode, if an instruction to switch to the standing position imaging mode or the sitting position imaging mode is input, the scanner 11 is tilted after rotating the couch 30 around the rotational axis R2 and moving the couch 30 from the imaging position B to the retraction position C.

In addition, in the standing position imaging mode or the sitting position imaging mode, if an instruction to switch to the recumbent position imaging mode is input, the scanner 11 is tilted and thereafter the couch 30 is rotated around the rotational axis R2 and is moved from the retraction position C to the imaging position B.

### Fifth Modification

Instead of moving the couch 30, the gantry 10 may be moved in accordance with the imaging mode. In this case, the moving mechanism 50 is attached to the gantry 10, and moves the gantry 10 along the moving direction. In the present modification, the moving mechanism 50 moves the gantry 10 in the major-axis direction (Z-axis direction, the imaging direction of the recumbent position imaging) of the couch 30. In the present modification, the major-axis direction (Z-axis direction, the imaging direction of the recumbent position imaging) of the couch 30 is the moving direction.

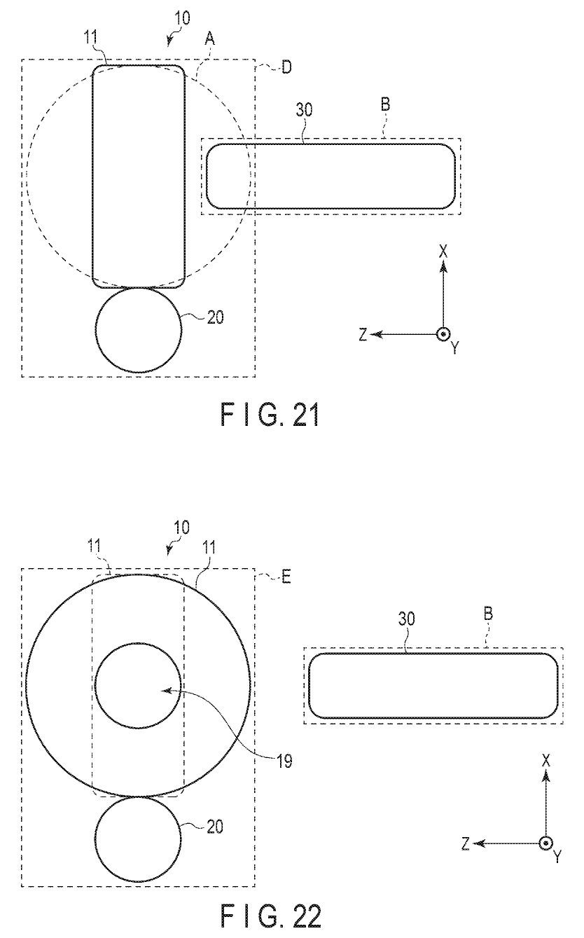

FIG. 21 is a diagram illustrating the positional relationship between the scanner 11 and the couch 30 in the recumbent position imaging mode, as viewed from above. FIG. 22 is a diagram illustrating the positional relationship between the scanner 11 and the couch 30 in the standing position imaging mode or the sitting position imaging mode, as viewed from above.

As illustrated in FIG. 21, in the recumbent position imaging mode, the scanner 11 is fixed in a state in which the center axis of the opening 19 is substantially parallel to the Z-axis direction. At this time, the gantry 10 is moved to an imaging position D. The imaging position D is preset at a position that is distanced from the couch 30 in the Z direction in the vicinity of the couch 30. In addition, the imaging position D is set at a position at which the top plate 33 can be inserted into the opening 19 and at which the movable range of the scanner 11 overlaps the imaging position B of the couch 30. This positional relationship is an example of the first positional relationship.

As illustrated in FIG. 22, in the standing position imaging mode or the sitting position imaging mode, the scanner 11 is fixed in a state in which the center axis of the opening 19 is substantially parallel to the Y-axis direction (vertical direction). At this time, the gantry 10 is moved to a retraction position E. The retraction position E is set at a position at which the movable range A of the scanner 11 does not overlap the imaging position B of the couch 30 and which is moved away from the couch 30 in the Z-axis direction, relative to the imaging position D. This positional relationship is an example of the second positional relationship.

In the present modification, in the recumbent position imaging mode, if an instruction to switch to the standing position imaging mode or the sitting position imaging mode is input, the scanner 11 is tilted after moving the gantry 10 from the imaging position D to the retraction position E along the Z-axis direction. In addition, in the standing position imaging mode or the sitting position imaging mode, if an instruction to switch to the recumbent position imaging mode is input, the scanner 11 is tilted and thereafter the gantry 10 is moved from the retraction position E to the imaging position D along the Z-axis direction.

Note that, instead of moving the gantry 10 in the Z-axis direction, the gantry 10 may be moved along the vertical direction (Y-axis direction) or along the width direction (X-axis direction) of the couch 30. Specifically, the moving direction of the gantry 10 by the moving mechanism 50 may be the vertical direction (Y-axis direction) or the width direction (X-axis direction) of the couch 30.

### Sixth Modification

In accordance with the imaging mode, the gantry 10 may be rotated around a rotational axis that is substantially parallel to the vertical direction. In the present modification, the rotational direction around the vertical direction is the moving direction.

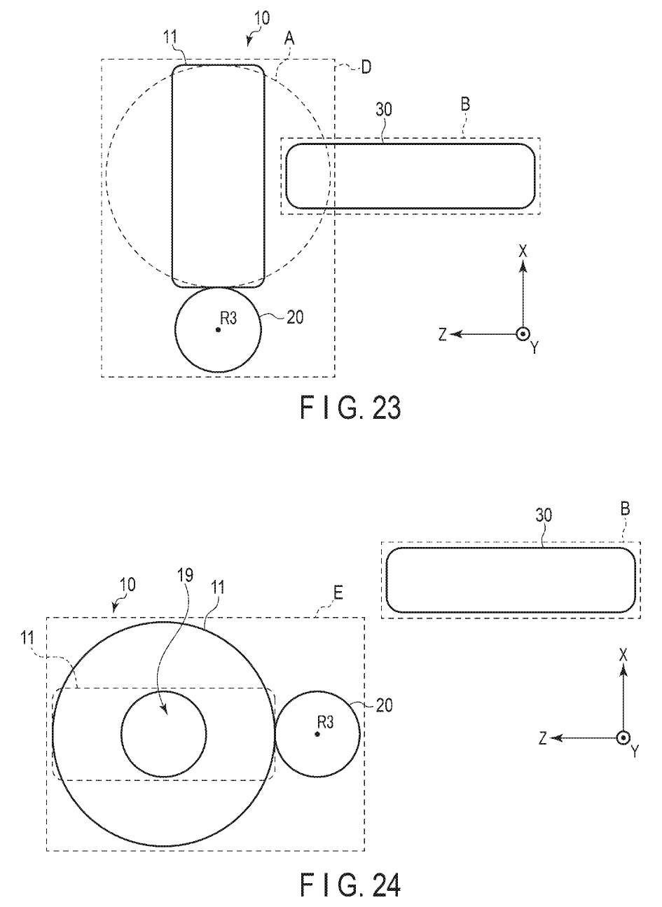

FIG. 23 is a diagram illustrating the positional relationship between the scanner 11 and the couch 30 in the recumbent position imaging mode, as viewed from above. FIG. 24 is a diagram illustrating the positional relationship between the scanner 11 and the couch 30 in the standing position imaging mode or the sitting position imaging mode, as viewed from above. In the standing position imaging mode and the sitting position imaging mode, the gantry 10 is moved to the retraction position E. The retraction position E is set at a position at which the movable range A of the scanner 11 does not overlap the imaging position B of the couch 30 and at which the gantry 10 is rotated by 90° around the center axis of the support column 20, relative to the imaging position D. This positional relationship is an example of the second positional relationship. The moving mechanism 50 is attached to the support column 20, and rotates the gantry 10 around a rotational axis R3 that is substantially parallel to the vertical direction.

In the present modification, in the recumbent position imaging mode, if an instruction to switch to the standing position imaging mode or the sitting position imaging mode is input, the scanner 11 is tilted after rotating the gantry 10 around the rotational axis R3 and moving the gantry 10 from the imaging position D to the retraction position E. In addition, in the standing position imaging mode or the sitting position imaging mode, if an instruction to switch to the recumbent position imaging mode is input, the scanner 11 is tilted, and thereafter the gantry 10 is rotated around the rotational axis R3 and is moved from the retraction position E to the imaging position D.

### Other Modifications

The moving mechanism may be provided to each of both the gantry 10 and the couch 30, and both the gantry 10 and the couch 30 may be moved in accordance with the imaging mode. The moving direction of the gantry 10 and the moving direction of the couch 30 may be identical or may be different.

For example, in addition to the moving mechanism 50 that moves the gantry 10, a second moving mechanism that moves the couch 30 is provided. The second moving mechanism may move the couch 30 in the same direction as the moving direction of the moving mechanism 50, or may move the couch 30 in a direction (second moving direction) different from the moving direction of the moving mechanism 50.

In the case where the moving mechanism may be provided to each of both the gantry 10 and the couch 30, the processing circuitry 44 moves the gantry 10 and the couch 30 such that the gantry 10 and the couch 30 approach each other, in a case where the mode switching function 443 switches to the recumbent position imaging mode using the couch 30, and moves the gantry 10 and the couch 30 such that the movable range of the scanner 11 does not overlap the position of the couch 30, in a case where the mode switching function 443 switches to the standing position imaging mode or sitting position imaging mode that does not use the couch 30. Thereby, the same advantageous effects as the above-described embodiments, etc., can be obtained.

### Common Configurations between Above-Described Embodiments and Modifications

The X-ray CT apparatus 1 of any one of the above-described embodiments and modifications includes the moving mechanism 50 and the control unit (for example, the processing circuitry 44 of the console 40). The moving mechanism 50 moves at least one of the gantry 10 and the couch 30 along the moving direction. Based on the first signal for the transition from the first positional relationship between the gantry 10 and the couch 30 at a time of executing the first imaging (for example, the recumbent position imaging) with use of the couch 30 to the second positional relationship between the gantry 10 and the couch 30 at a time of executing the second imaging (for example, the standing position imaging or sitting position imaging) with no use of the couch 30, the control unit (for example, the processing circuitry 44 of the console 40) tilts the scanner 11 and controls the moving mechanism 50 in such a manner that the gantry 10 and the couch 30 move away from each other. Alternatively, based on reception of the second signal for the transition from the second positional relationship to the first positional relationship, the control unit (for example, the processing circuitry 44 of the console 40) tilts the scanner 11 and controls the moving mechanism 50 in such a manner that the gantry 10 and the couch 30 approach each other.

According to at least one of the above-described embodiments, at a time of changing the imaging mode, a risk of interference between the couch and the gantry can be decreased.

The term "processor" used in the above description means, for example, a CPU, a GPU, or circuitry such as an application specific integrated circuit (ASIC), a programmable logic device (for example, a simple programmable logic device (SPLD), a complex programmable logic device (CPLD), or a field programmable gate array (FPGA)). The processor implements a function by reading and executing a program stored in storage circuitry. The program may be directly incorporated into the circuitry of the processor instead of being stored in the storage circuitry. In this case, the processor implements the function by reading and executing the program incorporated into the circuitry. The function corresponding to the program may be implemented by a combination of logic circuits, not by executing the program. Each processor of the present embodiment is not necessarily configured as single circuitry, but may include a plurality of units of independent circuitry to implement the functions of the processor. Furthermore, multiple components may be integrated into a single processor to implement the functions of the processor.

While certain embodiments have been described, these embodiments have been presented by way of example only, and are not intended to limit the scope of the inventions.

Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus comprising:
a gantry including a scanner with an imaging system, and a stand configured to tiltably support the scanner;
a couch including a top plate on which a subject is placed and a support frame configured to support the top plate so as to enable the top plate to move along a longitudinal direction; and
a control unit configured to perform positioning control such that
for executing first imaging which uses the couch, the scanner and the support frame are placed in a first positional relationship, and
for executing second imaging which does not use the couch, the scanner and the support frame are placed in a second positional relationship,
wherein, in the first positional relationship, a part of the support frame is located in a movable range of the scanner, and
in the second positional relationship, the support frame is located outside the movable range of the scanner.

2. The X-ray CT apparatus according to claim 1, further comprising a moving mechanism configured to move at least one of the gantry and the couch along a moving direction,
wherein the control unit is configured to execute either
a first control to tilt the scanner and control the moving mechanism such that the gantry and the couch move away from each other, based on a first signal for a transition from the first positional relationship for executing the first imaging to the second positional relationship for executing the second imaging, or
a second control to tilt the scanner and control the moving mechanism such that the gantry and the couch approach each other, based on a second signal for a transition from the second positional relationship to the first positional relationship.

3. The X-ray CT apparatus according to claim 2, wherein the moving direction is a direction along a major-axis direction of the couch.

4. The X-ray CT apparatus according to claim 2, wherein the moving direction is a direction along a width direction of the couch.

5. The X-ray CT apparatus according to claim 2, wherein the moving direction is a direction along a vertical direction.

6. The X-ray CT apparatus according to claim 2, wherein the moving direction is a rotational direction around a rotational axis extending along a vertical direction.

7. The X-ray CT apparatus according to claim 2, wherein the moving direction is a rotational direction around a rotational axis extending along a direction that is inclined with respect to a vertical direction and a major-axis direction of the couch.

8. The X-ray CT apparatus according to claim 2, wherein
the moving mechanism is configured to move the gantry along the moving direction, and
the X-ray CT apparatus further comprises a second moving mechanism configured to move the couch along the moving direction.

9. The X-ray CT apparatus according to claim 2, further comprising a second moving mechanism configured to move the couch along a second moving direction that is different from the moving direction,
wherein the moving mechanism is configured to move the gantry along the moving direction.

10. The X-ray CT apparatus according to claim 2, wherein in the first control, the control unit is configured to tilt the scanner after moving the gantry and the couch away from each other.

11. The X-ray CT apparatus according to claim 2, wherein in the second control, the control unit is configured to move the gantry and the couch toward each other after tilting the scanner.

12. The X-ray CT apparatus according to claim 2, wherein
the first imaging is imaging performed in a state in which a center axis of the scanner extends along a horizontal direction, and
the second imaging is imaging performed in a state in which the center axis of the scanner extends along a vertical direction.

13. The X-ray CT apparatus according to claim 2, wherein
the first imaging is recumbent position imaging, and
the second imaging is standing position imaging or sitting position imaging.

14. The X-ray CT apparatus according to claim 2, wherein the control unit is configured to control the moving mechanism in such a manner that the couch is located at a position overlapping a movable range of the scanner in the first positional relationship, and is configured to control the moving mechanism in such a manner that the couch is located at a position not overlapping the movable range of the scanner in the second positional relationship.

15. A control method of an X-ray CT apparatus comprising a gantry including a scanner with an imaging system, and a stand configured to tiltably support the scanner; a couch including a top plate on which a subject is placed; and a moving mechanism configured to move at least one of the gantry and the couch along a moving direction, the control method comprising:
executing either a first control to tilt the scanner and to control the moving mechanism in such a manner that the gantry and the couch move away from each other, based on a first signal for a transition from a first positional relationship between the gantry and the couch at a time of executing first imaging with use of the couch to a second positional relationship between the gantry and the couch at a time of executing second imaging with no use of the couch, or a second control to tilt the scanner and to control the moving mechanism in such a manner that the gantry and the couch approach each other, based on a second signal for a transition from the second positional relationship to the first positional relationship.
